(19) United States
(12) Patent Application Publication
(10) Pub. No.: US 2025/0000595 A1
(43) Pub. Date: Jan. 2, 2025

(54) MEDICAL ROBOT WITH INTUITIVE CONTROL, AND CONTROL METHOD

(71) Applicant: B. Braun New Ventures GmbH, Freiburg im Breisgau (DE)

(72) Inventors: Tim Beyl, Baden-Baden (DE); Shu Chuan Yao, Stuttgart (DE)

(21) Appl. No.: 18/709,080

(22) PCT Filed: Nov. 17, 2022
(86) PCT No.: PCT/EP2022/082334

§ 371 Date: May 10, 2024

(30) Foreign Application Priority Data
Nov. 18, 2021 (DE) 10 2021 130 238.2

(51) Int. Cl. A61B 34/37; A61B 34/00; A61B 34/30; A61B 90/00; A61B 90/25

(52) U.S. Cl. CPC A61B 34/37; A61B 34/74; A61B 90/25; A61B 2034/301; A61B 2034/742; A61B 2034/744; A61B 2090/064

(57) ABSTRACT

A medical robot for actuating a medical end effector includes a base; a movable arm connected to the base with at least one arm segment; an end effector connected to the arm with an end effector axis; and a control unit adapted to control the arm. The medical robot has an input mechanism with a neutral axis adapted to detect as input at least one force applied to the input mechanism transversely to the neutral axis in at least two opposite directions and convert the input to a control command for controlling a position and/or orientation of the end effector and/or the arm segment. An axis of the input mechanism is arranged parallel to the end-effector axis and/or to a longitudinal axis of the arm segment. A surgical assistance system, a control method and a computer-readable storage medium can be used with the robot.

### CROSS-REFERENCE TO RELATED APPLICATIONS

This application is the United States national stage entry of International Application No. PCT/EP2022/082334, filed on Nov. 17, 2022, and claims priority to German Application No. 10 2021 130 238.2, filed on Nov. 18, 2021. The contents of International Application No. PCT/EP2022/082334 and German Application No. 10 2021 130 238.2 are incorporated by reference herein in their entireties.

### FIELD

The present disclosure relates to a medical, in particular surgical, collaborative robot for actuating a medical end effector, in particular during a surgical intervention on a patient. The medical collaborative robot has a robot base as a local attachment point of the robot and, in a sense, as a local stationary coordinate system. Furthermore, the robot has a movable robot arm attached to the robot base; an end effector attached, in particular mounted, to the robot arm, in particular to an end side of the robot arm, the end effector having an end effector axis, in particular a visualization device as end effector, which has a (straight) visualization axis as end effector axis, and/or a medical, in particular surgical, instrument as end effector, which has an instrument axis as end effector axis at least at an end portion of the instrument; and a control unit adapted to actively control the robot arm in order to move the individual robot arm segments and thereby to adjust the position and/or orientation of the end effector. In addition, the present disclosure relates to a surgical assistance system, a control method and a computer-readable storage medium.

### BACKGROUND

In the field of medicine and medical technology, automation with the accompanying integration of digitally controllable technical devices is becoming increasingly important. Robots are increasingly being used for surgical interventions, in particular to support precise minimally invasive interventions. Here, the robot is not only intended as a stand-alone robot that solely performs the operation, but the robot is increasingly used as a collaborative robot (cobot), i.e. as an assisting or supporting robot, directly in the intervention field, which interacts with a medical professional, in particular the surgeon. For example, the collaborative robot can perform interventional surgeries in sections and hands over another interventional surgery to a surgeon as required.

In such an interaction between humans and robots, it is important to enable the user to control or guide the end effector or a part, a robot arm segment or an assembly of the robot via an input means/input device. In the current state of the art, operating buttons are provided for this purpose, wherein typically one button pair is used per axis in space, for example one button for movement along the X axis in the positive direction and one button for movement along the X axis in the negative direction. The buttons may be provided in an external control console or at another location where the user interacts with the robot, in the manner of a remote control. However, in cases where the input device is located somewhere other than on the moving segments of the robot, haptic feedback from the robot controller is completely lost.

In addition, the use of operating buttons requires the surgeon to perform a high level of mental connection between a coordinate system of the end effector and a coordinate system of the control device with the operation keys, which is tiring and increases the susceptibility to errors during the intervention. Although it is currently possible to display an extracorporeal image of the surgical environment or of the surgical intervention area on a separate monitor and to control the robot on the basis of the extracorporeal image, this requires the surgeon to constantly move the head, which is tiring. Furthermore, such a modality is not suitable for rough positioning of the end effector and a user easily loses the spatial overview. There is also the mental challenge and effort required for orientation while mentally merging the information, in particular the position and orientation of the end effector relative to the patient. This poses a great challenge, in particular for those involved in surgery who do not have a sufficiently well-developed spatial imagination for these purposes, and makes it difficult to link information.

Another approach is to build a torque sensor into a hinge of a robot, which makes it possible to measure the torque per hinge and drive. Using information about the kinematics and dynamics of the robot as part of a mathematical model, an interaction force on the end effector of the robot can be calculated in most scenarios, so that a robot with hinge torque sensors can also perform control in special scenarios. Torque sensors in the hinges also allow the user to interact with other parts of the robot. Typically, robots with hinge torque sensors have a control mode in which the user can apply a force to a hinge and the hinge(s), in which the resulting torque is measured, follows the torque using a control algorithm that translates the torque into a resulting motion. However, this approach has the major disadvantage that unintentional contact with a part of the robot can trigger an unintentional and uncontrollable movement, which in the worst case may have lethal consequences for a patient.

### SUMMARY

It is therefore the object of the present disclosure to avoid or at least reduce the disadvantages of the prior art and in particular to provide a medical, preferably a surgical, collaborative robot (cobot), a surgical assistance system, a control method and computer-readable storage medium which allows interaction with a user and control by a user in a particularly simple, intuitive but also safe manner. A partial object can be seen in allowing only a limited, selected movement of an end effector or a robot-arm segment or an assembly group of the robot, if required, in order to enable precise control during an intervention and to strictly prevent unwanted, disadvantageous movements of the robot.

The objects are solved with respect to a medical, in particular surgical, collaborative robot (cobot), are solved with respect to a generic surgical assistance system, are solved with respect to a generic control method, and are solved with respect to a computer-readable storage medium.

A basic idea is therefore to provide at least one operating element (an input means and/or detection means or operating means and/or recognition means) in a (collaborative) robot with an articulated robot arm and an end effector connected to the robot arm, at a point of the robot arm or end effector that moves with the end effector and/or the robot arm segment of the robot arm and, in addition, is also specifically aligned with an axis of the end effector and/or with an axis of the associated robot arm segment of the robot arm, i.e. parallel, in particular coaxially, to the corresponding axis, in order to enable particularly intuitive and safe control.

In other words, the present disclosure thus describes in particular a targeted arrangement of special interaction units (input means and/or detection means), in particular haptic interaction units, on the robot arm of the robot, i.e. on the structure of the robot, and/or on the end effector, in order to be able to interact with the collaborative robot in a particularly advantageous and intuitive manner. This special placement and alignment of the haptic interaction device on the end effector and/or on the structure of a robot, in particular on the robot arm segment, guarantees the user intuitive haptic guidance of the end effector and/or of a robot segment or a robot part by the input device/input means.

In yet other words, a core of the present disclosure is a targeted alignment of a specific axis (in particular the neutral axis) of the input means relative to an axis of the at least one end effector (end-effector axis) and/or of the robot-arm segment (longitudinal axis of the robot-arm segment or of a straight extension of the longitudinal axis of the robot-arm segment toward the input means), wherein an axis of the end effector or the longitudinal axis of the robot-arm segment is parallel to this specific axis (in particular neutral axis) of the input means, in particular of a neutral axis of the joystick. A robot or robot system is therefore proposed that enables the user to intuitively move the robot arm and/or the end effector into the target position or target orientation. This is realized in particular with an input means, preferably a joystick, which is attached in particular to an intervention and/or visualization device, i.e. is connected to the end effector, wherein an axis, in particular a neutral axis of the input means runs parallel, in particular coaxially, to an intervention or visualization axis.

In even other words, the present disclosure proposes a medical, in particular surgical, collaborative robot for actuating a medical, in particular surgical, end effector, comprising: a robot base as a local attachment point of the robot; a movable robot arm connected to the robot base and having at least one robot arm segment; an end effector connected, in particular mounted, to the robot arm, in particular to an end side of the robot arm, and having an end effector axis, in particular a visualization device, having a visualization axis, and/or a medical, in particular surgical, instrument having an instrument axis; and a control unit adapted to control at least the robot arm. It is special here that the medical robot, in particular on its end effector and/or on a robot arm segment, has at least one input means with an, in particular straight, neutral axis which is adapted to detect at least one force (manually) applied to the input means transversely to the neutral axis in at least two opposite directions and to act as a control command of the control unit for controlling a position and/or orientation of the end effector (if an input means is provided on the end effector) or respectively of the robot-arm segment (if an input means is provided on the robot-arm segment), and an axis of the input means, in particular the neutral axis of the input means, is arranged substantially parallel, in particular coaxially, to the end-effector axis (if an input means is provided on the end effector) and/or to a longitudinal axis of the robot-arm segment (if an input means is provided on the robot-arm segment). In particular, the neutral axis is arranged parallel, in particular coaxial, to the visualization axis and/or to the instrument axis.

The term ‘neutral axis’ defines an axis of a neutral position or inoperative state of the input means in which no manually applied (input) force acts on the input means. In particular, the neutral axis is a type of symmetry axis of the input means, which allows at least one input in two opposite directions transverse to the neutral axis. In particular, the neutral axis serves to orient an input of the input means.

In the present disclosure, the term ‘end effector’ means, for example, a device, instrument or similar medical means that can be used to perform an intervention on a patient. In particular, an end effector may be considered to be: an instrument, a medical device such as an endoscope or suction tube, an optical device with a visualization axis, a pointer/indicator with a distal tip for surgical navigation, and other devices. The term ‘distal tip’ describes the distal area of the pointer, which, starting from a basic shape, tapers at a distance perpendicular to the longitudinal axis, in particular in diameter, and forms a distal end face for palpation of the tissue via the continuous tapering.

The term ‘robot arm segment’ here means in particular a robot part of the robot arm mounted between hinges. A longitudinal axis means in particular a portion of the longitudinal axis adjacent to the input means, i.e. the portion at the input means.

The term ‘position’ means a geometric position in three-dimensional space, which is specified in particular via coordinates of a Cartesian coordinate system. In particular, the position can be specified by the three coordinates X, Y and Z.

The term ‘orientation’ in turn indicates an alignment (for example at the position) in space. It can also be said that the orientation indicates an alignment with an indication of direction or rotation in three-dimensional space. In particular, the orientation can be specified using three angles.

The term ‘pose’ includes both a position and an orientation. In particular, the pose can be specified using six coordinates, three position coordinates X, Y and Z and three angular coordinates for the orientation.

In the present case, the term ‘axis’ means an axis of a (in particular Cartesian) coordinate system of the input means with three axes that all intersect at one point, with one axis in particular being the ‘neutral axis’.

The present disclosure is also not limited to attaching the input means directly to the end effector, but it may also be provided at a location where the axis, in particular neutral axis, of the input means is parallel to an interaction axis of the end effector (for example to a visualization system or to an instrument guide).

Advantageous embodiments are claimed in the dependent claims and are explained in particular below.

Preferably, the input means may have a deflectable lever with a longitudinal lever axis, which can be deflected about a deflection point located on the neutral axis relative to the neutral axis with a deflection angle, and which pivots back into its neutral position relative to the neutral axis without application of force. In particular, the input means is configured as a joystick in order to detect an input in at least four directions (one plane). In the case of a lever, in particular a joystick, a force perpendicular to the neutral axis (outside the deflection point) can be used to cause a movement of the lever in the direction of the force. This movement is detected by sensors and is provided to the control unit as a control command. In particular, the deflection angle correlates with an amplitude of an input and a resulting movement speed of the end effector or of the robot arm segment. Preferably, the medical robot therefore has, in particular, a control device (with corresponding control algorithms) and an input means, in particular a joystick, which is preferably attached to the end effector of the robot, with at least one axis (neutral axis) of the input means, in particular the joystick, running parallel to at least one axis of the end effector. A robot system is thus provided that enables the user to intuitively move the robot arm with the end effector into the desired position, in particular pose. This is realized in particular by an input means, in particular a joystick, which is attached to the end effector, in particular to an intervention device or visualization device, wherein an input means axis/neutral axis, in particular a neutral axis of the joystick, is aligned parallel to the end effector axis, in particular to the intervention axis and/or visualization axis. The robot thus has, in particular at its end effector, an input means which has a deflectable lever with an adjustable deflection angle relative to a deflection point, in particular in the form of a joystick, the neutral axis being (always) aligned/arranged parallel, in particular coaxially, to the end effector axis, in particular to the visualization axis and/or to the instrument axis.

In particular, a joystick is used as an input means, which is attached to the robot flange or end effector, wherein the movement of the joystick is used as input for the controller, which generates the movement signals to be followed by the robot. One idea is to simulate the movement of an end effector by using a joystick. Typically, a joystick has a spring-like behavior. In particular, the joystick is mounted on the end effector, wherein the end effector and the joystick each have their own reference system. The joystick itself has a deflection point around which all axes rotate. This rotation point/deflection point, including the alignment of the joystick, is then translated in particular into the target coordinate system, which may be a remote frame (e.g. the point around which a microscope focuses). A special feature is that the remote frame, which is controlled with the joystick, moves together with the joystick and the robot. A special property here is that one axis of the joystick, i.e. the neutral axis, is parallel to an axis of the controlled frame. This means that at least one axis of the joystick and the robot have the same alignment. In the case of a microscope, for example, this has the effect that the joystick grips around the optical axis of the optical system and moves this optical axis accordingly. In particular, at least one axis of the end effector is aligned with an axis of the joystick, in this case the neutral axis.

In particular, the input means, in particular the joystick, is not detachably connected to the end effector and/or to the robot arm and is thus to a certain extent an integral part of the robot, in particular of the end effector.

According to an embodiment, a 3D mouse/a (3D) space mouse/a 3D space mouse may be used as the at least one input means, which, in addition to three axis inputs (up/down, front/back, left/right or in X-Y-Z of a Cartesian KOS), also enables three rotation inputs (around the three axes in each case), wherein one axis of the 3D mouse, in particular a Z axis, is aligned as a neutral axis parallel, in particular coaxially, to the end-effector axis and/or to the longitudinal axis of the robot arm segment, in order to move the end effector (if parallel to the end-effector axis) and/or the robot-arm segment (if parallel to the longitudinal axis of the robot-arm segment) translationally and rotationally (in particular around a focal point or an instrument tip) in accordance with the input. In an embodiment, a 3D spacemouse may therefore be used as a joystick to control the robot. An example of a 3D mouse is a 3D mouse marketed by 3Dconnexion under the registered trademark SPACEMOUSE® Compact (with Z axis perpendicular to the support surface).

According to a further preferred embodiment, a force-momentum sensor/force-torque sensor may be used as the at least one input means, which has six input-degrees of freedom with three axis inputs and three rotation inputs. One way to interact with the end effector is to use a force-torque sensor, which is attached to the end effector and/or the robot arm segment and measures the interaction forces between the user and the robot with which he interacts haptically. A controller typically uses a dynamic model of the robot to generate a movement signal for the robot so that the robot follows the interaction forces. As an alternative or in addition to the 3D spacemouse, a force-torque sensor may also be used, which in particular is attached to the end effector, to a handle and/or to a robot arm segment. A neutral axis is substantially parallel, in particular coaxial, to an end-effector axis or to the axis of the robot arm segment on which the force-torque sensor is provided.

Preferably, the medical robot may have a (surgical) microscope as a visualization device, which has/has set/comprises a focal point on its visualization axis. Preferably, the control unit is adapted, in case of an input by the input means, to control the robot arm with the microscope head (according to the controller) in such a way that the position of the focal point (relative to the patient) is maintained and the focal point remains on the visualization axis. In particular, the robot can thus resemble a surgical microscope, wherein the microscope is preferably controlled by joystick movements

According to an embodiment, the robot may have an instrument with an instrument tip on the instrument axis as the end effector, and the control unit may be adapted to control the robot with the instrument in such a way that the position of the instrument tip is maintained during an operating input by the input means. This configuration of the control unit allows the orientation of the instrument to be changed while the distal tip of the instrument remains at its target position or setpoint position during an intervention.

According to a further embodiment, the input means may be adapted to select an end effector frame and/or a target frame and/or a base frame and/or a world frame and/or a frame defined by the user and/or an image frame as a frame and to control it accordingly. The input means, in particular the 3D mouse/3D spacemouse, may thus be adapted to control the robot, in particular the end effector, in a target frame, a base frame, a world frame, a user-defined frame or in an image frame, in particular when a camera is mounted on the robot or used in combination with the robot (e.g. mounted on an external assembly group). In particular, the 3D mouse is used to control the robot in an end effector frame.

In particular, the input means may have six degrees of freedom (6DoF), wherein (three) translational axes of the input means, in particular of the 3D mouse, control the (three) translational movements of the robot in the selected frame, and (three) rotational axes of the input means control (three) rotational movements of the robot in the selected frame. In particular, the 3D spacemouse may be used in a mode with six degrees of freedom (6-Dof mode), in which the translational axes of the 3D spacemouse control the translational axes of a translation performed by the robot in the frame/framework in which the 3D spacemouse manipulates the robot and the rotational axes of the 3D spacemouse control the rotations performed by the robot in the frame/framework in which the 3D spacemouse manipulates the robot.

Furthermore, in particular the control unit may allow exactly three degrees of freedom (3 DoF) of the input means as input, wherein either
(three) translational axes of the input means, in particular of the 3D mouse, control the (three) translational movements of the robot in the selected frame, or
the (three) rotational axes of the input means control (three) rotational movements of the robot in the selected frame. In particular, the 3D spacemouse may therefore be limited to a mode with three degrees of freedom (3DoF mode), in which either translations or rotations are controlled and the remaining other degrees of freedom are not processed in the control unit.

According to a further embodiment, the control unit may be adapted to control a single degree of freedom or a combination of degrees of freedom of the robot, in particular the end effector and/or of the robot arm segment, on the basis of a (stored) number of degrees of freedom between one and five. In other words, the control unit may be adapted to block one to five degrees of freedom or not take them into account when controlling the robot arm. In particular, the 3D spacemouse may be limited to any number of degrees of freedom (DoF) from one to five to allow manipulation of a single degree of freedom or a combination of degrees of freedom in the selected frame and/or task space of the robot.

Preferably, the end effector may further comprise a camera, in particular a wide-angle camera, with a camera axis, wherein the camera axis is aligned/arranged parallel, in particular coaxially, to the end effector axis, in particular to the visualization axis and/or instrument axis. In this way, an overview and an optical visualization are provided. In particular, the assembly group of the end effector is therefore supplemented by a camera on the end effector, with an optical axis running parallel to at least one axis of the input means, in particular of the joystick. Preferably, the optical axis of the camera may be aligned parallel, in particular coaxially, to a (neutral) axis of the joystick.

According to a further embodiment, the input means may have a puck-shaped or ball-shaped or cube-shaped (manually operable) haptic outer contour as an operating unit. In particular, the input means is a puck-shaped joystick with preferably six degrees of freedom, which is attached to the end effector and/or robot segment. Alternatively, the haptic user interface may have the shape of a sphere or a cube.

According to a preferred embodiment, the (analog) input signal, for example proportional to a deflection of the joystick lever, may be used to modulate the speed of movement of the hinges.

Preferably, the robot may have a distance sensor in order to control the robot arm via the control unit so that it avoids objects that come close to the sensor. Preferably, the distance sensor may be used with a threshold value so that the robot kinematics are only optimized when an object is below a certain distance.

According to one embodiment, the input means, in particular the joystick, may have a sterile cloth or sterile cover, in particular may be surrounded by a sterile cloth or sterile cover, in particular may be enwrapped by it. Preferably, the sterile cloth is arranged in such a way that it does not touch the joystick, so that neither force nor torque is exerted on the joystick.

Preferably, the joystick may be enwrapped with a sterile cloth or a sterile cover that exerts a force and/or torque on the joystick. The control unit is adapted to use an algorithm to distinguish between forces and torques exerted by the cloth or cover and forces and torques exerted by a user in order to compensate for the forces and torques exerted by the sterile cloth.

In particular, the last robot-arm segment, i.e. the last robot-arm segment before the end effector, has the input means, in particular the joystick.

Preferably, in the case of a surgical microscope as end effector and an instrument as end effector, the 3D spacemouse can be used to switch back and forth between a fixed-position focal point of the surgical microscope and a fixed-position instrument tip via the operation of a switch or a button or an operation surface. In this way, a movement around either the focal point or the instrument tip can be realized with only one input means.

With regard to a surgical assistance system for use in a surgical intervention on a patient, the objects are solved in that this assistance system comprises a surgical robot according to the present embodiment for intuitive control and a navigation system for navigation during the intervention. In this way, the control of the robot together with the surgical navigation system can be performed even better and more intuitively.

Preferably, the assistance system may be adapted via the control unit to move the end effector at a higher speed when the distance between the end effector and the patient is above a distance threshold when input is provided via the input means than when the distance is less than the distance threshold, in order to increase safety.

According to an embodiment, the assistance system may be adapted to limit a degree of freedom in a movement of the end effector based on navigation data. For example, the assistance system may only allow movement along an opening channel during a minimally invasive intervention and block rotational movement and other translational movement.

In particular, the surgical assistance system may be adapted to move the end effector to a selected point and then transfer control to the user so that the user can intuitively control the end effector using the input means.

The objects of the present disclosure are solved with respect to a control method for a surgical robot, in particular according to the one of the present disclosure, in that the control method comprises the steps of: detecting an input by an input means whose neutral axis is aligned parallel, in particular coaxially, to an end-effector axis of an end effector and/or to a longitudinal axis of a robot-arm segment and providing the detected input to a control unit; and controlling a position and/or orientation of the end effector and/or of the robot-arm segment by the control unit on the basis of the detected input.

With regard to a computer-readable storage medium or a computer program, the objects are solved by comprising instructions which, when executed by the computer, cause the computer to perform the method steps of the control method according to the present disclosure.

Any disclosure related to the collaborative robot according to the present disclosure applies to the control method according to the present disclosure as well as vice versa.

### BRIEF DESCRIPTION OF THE DRAWINGS

The invention is explained in more detail below with reference to preferred embodiments with the aid of Figures.

FIG. 1 shows a perspective view of a surgical collaborative robot with a medical instrument and a surgical microscope according to a first preferred embodiment, which is controlled by a 3D spacemouse;

FIG. 2 shows a perspective view of a collaborative robot with a camera as end effector according to a further, second preferred embodiment, in which a 3D spacemouse controls the pose of the camera and a 3D spacemouse controls a robot arm segment;

FIG. 3 shows a schematic view of a joystick as an input means for a robot according to the present disclosure;

FIG. 4 shows a schematic view of a force-momentum sensor as an input means for a robot according to the present disclosure;

FIG. 5 shows a perspective view of a surgical collaborative robot with a medical instrument and a surgical microscope according to a further, third preferred embodiment, which is controlled by a 3D spacemouse; and

FIG. 6 shows a flowchart of a control method according to a preferred embodiment for controlling a medical collaborative robot.

The Figures are schematic in nature and are only intended to aid understanding of the invention. Identical elements are marked with the same reference signs. The features of the various embodiments can be interchanged.

### DETAILED DESCRIPTION

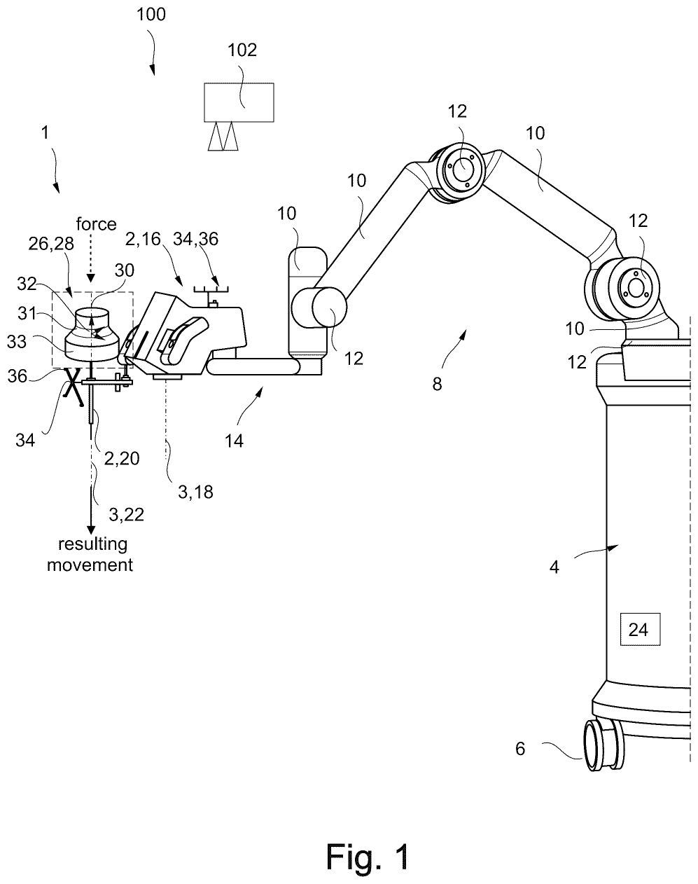

FIG. 1 shows a perspective view of a surgical collaborative robot 1 (hereinafter referred to as cobot) according to a first preferred embodiment for actuating an end effector 2. The cobot 1 is part of a surgical assistance system 100 which enables surgical navigation in order to guide the end effector 2 in a targeted manner. In this variant, the cobot 1 has a robot base 4 that is configured in the form of a cart with rollers 6 in order to make the robot mobile so that it can be used as required at different locations in an operating theater in a hospital. The robot base 4 forms a local reference point of the cobot 1, and a multi-hinged/multi-jointed/multi-segmented robot arm 8 with several robot arm segments 10, which are connected to each other via hinges 12, is attached to an upper side facing away from the rollers 6. In this way, the robot arm segments 10 can be actively moved in relation to each other and the robot arm 8 can be controlled as a whole.

The end effectors 2 are attached to a flange of the robot arm 8 on an end side or end portion 14 of the robot arm 8. One end effector 2 is a surgical microscope 16 with a visualization axis 18 and the other end effector 2 is a medical instrument 20 rigidly arranged for this purpose with an instrument axis 22. The position and orientation, i.e. the pose, of both the surgical microscope 16 and the medical instrument 20 can be controlled and adjusted by the robot arm 8. A specially adapted control unit 24, which is provided on the robot base 4, serves as the central control unit for this purpose.

In contrast to the prior art, the cobot 1 has a 3D spacemouse 28 on its end effector 2 or its end effectors 2 as input means 26 for control and is adapted to receive both translational control command inputs for three axes 32 that are perpendicular to each other and rotational control command inputs about these three axes 32 manually by haptic operation and to forward them to the control unit 24 as computer-readable, digital control signals or control commands, so that the control unit 24 can actively control the robot arm 8 in accordance with the control commands.

Specifically, the 3D spacemouse 28 allows manual input in a longitudinal direction, which also represents its axis of symmetry of the haptic outer surface of an operating portion 31 and can be (easily) moved relative to an operating base 33 in the manner of a joystick, wherein the axis of symmetry also represents the neutral axis 30 of the 3D spacemouse (seen in FIG. 1, an axis in the up-down direction). In an undeflected position of the operating portion 31, the axis of symmetry lies on the neutral axis 30. The neutral axis 30 defines, so to speak, a possible axis of the operating portion 31, in which (apart from a possible coaxial input) no input signal occurs and forms an inoperative state, so to speak. If the operating portion 31 of the 3D spacemouse 28 is manually pulled upward along the neutral axis 30 (application of traction by a surgeon), the 3D spacemouse 28 detects this input and forwards it to the control unit 24. On the other hand, the 3D spacemouse 28 can be manually pushed downward (as seen in FIG. 1) along the neutral axis 30 (application of pressure), and the pressure input can be detected by the 3D spacemouse 28. In the present case, the axis 32, which is coaxial to the neutral axis 30 in the inoperative state/neutral position, is referred to below as the Z axis for better understanding.

Similarly, the 3D spacemouse 28 allows a (control command) input in this plane by applying a pull or pressure in a plane perpendicular to the neutral axis 30, so that control command inputs can be made in three axes 32 that are perpendicular to each other, which the control unit 24 detects as movement command inputs and interprets as translational command inputs. It can also be said that a local coordinate system (KOS) of the operating portion 31 is changed, i.e. shifted or rotated, in relation to the local coordinate system (KOS) of the base portion 33 by an input. This new transformation (compared to the old transformation) between the two local coordinate systems is recorded as a control command input and forwarded to the control unit 24.

In addition, the 3D spacemouse 28 can be pivoted or rotated about all three axes 32 as a kind of (joystick) lever, so that in addition to the above control command inputs along the three axes 32, rotational control command inputs are also detected. The 3D spacemouse therefore has a total of 6 degrees of freedom (6DoF), three translational degrees of freedom (3DoF) and three rotational degrees of freedom (3DoF).

Furthermore, in contrast to the prior art, the 3D spacemouse 28 is arranged specifically with respect to the end effector 2 and its end effector axis. Concretely, in this embodiment, the 3D spacemouse 28 is arranged specifically with respect to the instrument axis 22 of the instrument 20. The neutral axis 30 of the 3D spacemouse 28, i.e. the Z axis of the axes 32 in the inoperative state, is aligned coaxially with the instrument axis 22.

In this way, a user or surgeon receives a unique intuitive control option with direct, immediate movement feedback when operating the end effector 2 using the input means 26 in the form of the 3D spacemouse 28. If the surgeon presses the 3D spacemouse 28 downward (in the alignment of the end effector 2 shown in FIG. 1) along the neutral axis 30 (or the Z axis in the inoperative state), the 3D spacemouse 28 detects the input and forwards it to the control unit 24. The control unit 24 processes the control command input and then controls the robot arm 8 in such a way that the end effector 2 is moved downward along the neutral axis 30 or Z axis, which is also the instrument axis 22 as seen in FIG. 1. Pressure in the desired direction thus causes the instrument 20 to move in precisely this desired direction. Likewise, a push (or pull) in a direction transverse to the neutral axis 30 also causes the instrument 20 to move in precisely this direction. Since the operating portion 31 can also be pivoted or rotated around the axes like a joystick, a surgeon can also pivot the instrument 20 around this axis.

The cobot 1 with the neutral axis 30 arranged coaxially to the instrument axis 22 allows the surgeon to control the end effector 2 particularly intuitively and easily. Any mental effort required for a geometric correlation of input to movement of the end effector is reduced to a minimum, which reduces fatigue of the surgeon and further increases safety during an intervention.

In this embodiment, the visualization axis 18 is especially also aligned parallel to both the instrument axis 22 and the neutral axis 30. Due to the rigid arrangement, all axes 18, 22 and 30 remain parallel to each other at all times. This means that the surgeon can not only guide the instrument 20 particularly well, but also the head of the surgical microscope 16.

In an alternative embodiment, not shown here, the input means may also have a coaxial arrangement of neutral axis 30 and visualization axis 18 instead of or in addition to the coaxial arrangement of neutral axis 30 to instrument axis 22.

Both the instrument 20 and the head of the surgical microscope 16 are fitted with infrared markers or trackers 34, each with four spaced-apart infrared spheres 36, for surgical navigation by the surgical assistance system 100 with a tracing apparatus 102 in the form of a 3D camera, in order to detect and track the pose of both the instrument 20 and the head of the surgical microscope 16.

As explained above, the 3D spacemouse 28 allows control command inputs with six degrees of freedom. However, the control unit 24 also allows degrees of freedom to be blocked so that a selection can be made as to which translational and/or rotational movements are permitted. For example, the user can specify that they only want to make a translatory movement in the Z axis direction, i.e. along the neutral axis 30, and the control unit 24 blocks the remaining five degrees of freedom. With an input by the 3D spacemouse 28, ‘several’ control command inputs can be detected, but only the control command input in the direction of the Z axis is selected and the robot arm 8 with the instrument 20 is moved accordingly along the Z axis. In this way, unwanted movement is prevented by an unwanted input in a different direction of the sensitive 3D spacemouse 28.

The control unit is also adapted to lock the translational degrees of freedom and to control the robotic arm 8 during an input in such a way that a position of an instrument tip of the instrument 20 is maintained and the instrument 20 moves around the fixed point of the instrument tip in the manner of a joystick.

The instrument tip of the instrument 20 may also be used to mark a point in space on the patient for the surgical assistance system 100, in particular for accurate localization and registration.

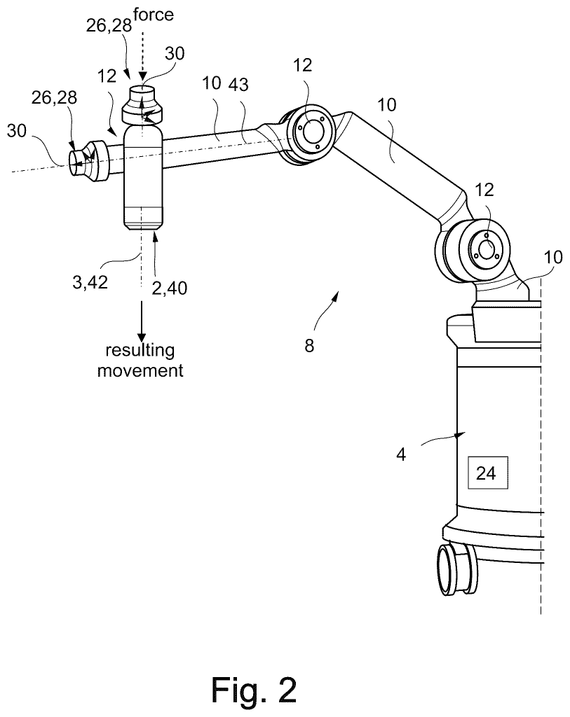

FIG. 2 shows a cobot 1 according to a further, second preferred embodiment. In contrast to the first preferred embodiment described above, the cobot 1 has a 3D camera 40 with visualization axis 42 as end effector 2, the pose of which can be adjusted via the robot arm 8 via an input from the (first) 3D spacemouse 28, which is arranged coaxially to the visualization axis 42.

Furthermore, not only is an input means 26 provided on the end effector 2, but a second input means 26 in the form of a second 3D spacemouse 28 is also provided. This second 3D spacemouse 28 is arranged coaxially to the longitudinal axis of the robot arm segment 10 directly adjacent to the end effector 2. The pose of the corresponding last robot arm segment 10 can be explicitly set via this second 3D spacemouse 28. The control unit 24 is adapted to control the robot arm segment 10, analogous to the input described for the first embodiment. A manually applied pressure in the direction of the neutral axis 30 of the second 3D spacemouse 28 causes a translational movement of the (last) robot arm segment 10 in the direction of the pressure (it adapts to the pressure, so to speak) according to the freedom of movement of the robot arm 8. The control unit 24 is furthermore adapted to move the robot arm while an orientation or alignment of the 3D camera 40 is maintained and only a position of the 3D camera 40 changes. Since the 3D camera 40 is connected to the robot arm segment via a hinge, this allows for corresponding kinematics. While the pose of the 3D camera 40 can be controlled intuitively with the first 3D spacemouse 28, the pose of the (last) robot arm segment 10 can be controlled intuitively with the second 3D spacemouse 28.

At this point, it should be noted that the operating portion 31 does not necessarily have to move relative to the base portion 33, since input means with pressure sensors and torque sensors are also conceivable, which can detect a control command input by the user. This is realized in particular by a force-momentum sensor as input means (which also has a neutral axis). In this way, moving parts can be further reduced and gaps or cracks can be avoided for better sterilizability.

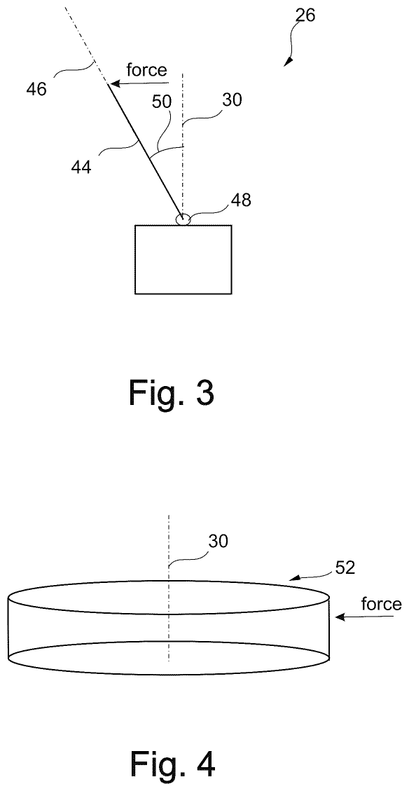

FIG. 3 shows a schematic view of a joystick as input means 26 for a medical collaborative robot 1 according to the present disclosure. The joystick has a lever 44 with a longitudinal lever axis 46, which can be deflected relative to a deflection point 48 by a deflection angle 50. According to the deflection angle 50, an amplitude of the input is detected and forwarded accordingly as a control command to the control unit 24. The (joystick) lever 44 is elastically moved back to its neutral position without the application of force by a user, in particular a surgeon. In this neutral position, the longitudinal lever axis 46 lies on the neutral axis 30.

FIG. 4 shows a schematic view of a force-momentum sensor 52 as an input means 26 for a medical collaborative robot 1. The force-momentum sensor 52 has a neutral axis 30 that is coaxial to a (translational) axis. In this embodiment of the input means 26, pressures and torques are detected via sensors, so that there is no movement as with a joystick. This has the advantage that there is no geometric movement of an operating portion relative to an operating base and all can be configured as a closed geometric system.

The force-momentum sensor 52 can be used instead of the 3D spacemouse 28 in the cobot 1 of FIG. 1 or 2.

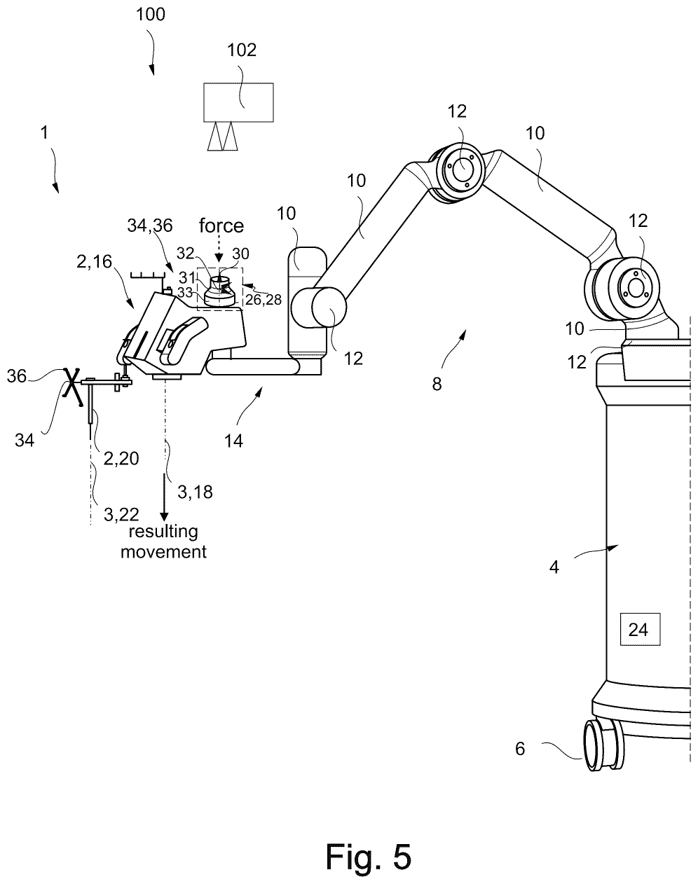

FIG. 5 shows a collaborative robot 1 according to a further preferred embodiment, which is controlled by a 3D spacemouse 28. In contrast to the first embodiment, the 3D spacemouse 28 is arranged in the area of the surgical microscope 16 (as robot head), no longer coaxial to the end-effector axis 3, but parallel to the visualization axis 18 of the surgical microscope 16 and thus, in this embodiment, also parallel to the instrument axis 22.

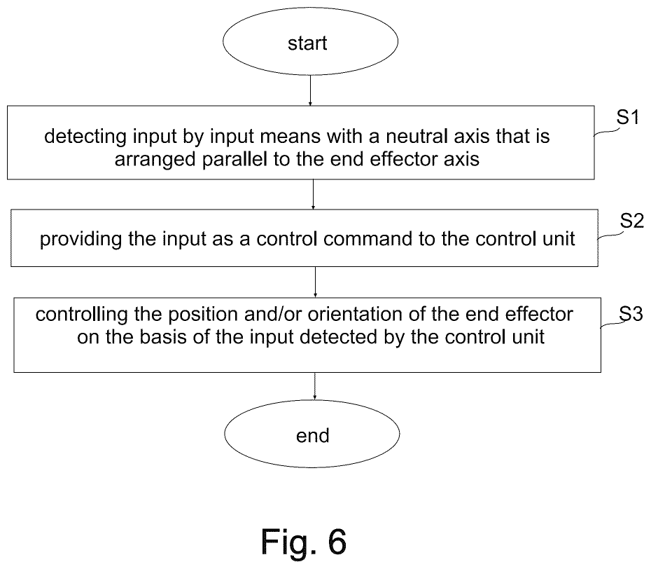

FIG. 6 shows a flow diagram of a control method of a preferred embodiment.

In a first step S1, an input is detected by an input means 26 whose neutral axis 30 is aligned parallel, in particular coaxially, to an end effector axis 3 of an end effector 2.

In a second step S2, the detected input is provided as a control command to a control unit 24. The control unit 24 processes the control command accordingly.

In a step S3, a position and/or orientation of the end effector 2 is then controlled by the control unit 24 on the basis of the detected input.

According to a further preferred embodiment, the control method may further comprise the step of blocking a degree of freedom by the control unit 24 and controlling the end effector 2 by the control unit 24 based on the remaining degrees of freedom. intentionally left blank

### LIST OF REFERENCE SIGNS

1 medical collaborative robot/cobot
2 end effector
3 end-effector axis
4 robot base
6 rollers
8 robot arm
10 robot-arm segment
12 hinge
14 end portion robot arm
16 surgical microscope
18 visualization axis of surgical microscope
20 medical instrument
22 instrument axis
24 control unit
26 input means
28 3D Spacemouse
30 neutral axis
31 operating portion
32 axes
33 base portion
34 tracker
36 infrared spheres
40 3D camera
42 visualization axis of 3D camera
43 longitudinal axis robot-arm segment
44 lever
46 longitudinal lever axis
48 deflection point
50 deflection angle
52 force-momentum sensor
100 surgical assistance system
102 tracing apparatus
S1 Step detecting input by input means with parallel neutral axis to end-effector axis
S2 step providing input as control command to control unit
S3 Step controlling pose of end effector

1.-15. (canceled)

16. A robot configured to be a medical collaborative robot for actuating a medical end effector, the robot comprising:
a robot base as a local attachment point of the robot;
a robot arm that is movable and connected to the robot base and having at least one robot arm segment;
a control unit adapted to control the robot arm; and
an input mechanism,
the medical end effector connected to the robot arm and having an end effector axis;
the input mechanism having a neutral axis and being adapted to detect as input at least one force applied to the input mechanism transversely to the neutral axis in at least two opposite directions and to provide a control command of the control unit for controlling a position and/or orientation of the medical end effector and/or of the at least one robot arm segment, and
an axis of the input mechanism being arranged coaxially with the end effector axis.

17. The medical robot according to claim 16, wherein the input mechanism has a deflectable lever with a longitudinal lever axis, the deflectable lever being deflectable about a deflection point located on the neutral axis relative to the neutral axis with a deflection angle, the deflectable lever being pivotable back into a neutral position coaxially to the neutral axis without application of force, wherein the input mechanism is configured as a joystick to detect an input in at least four directions.

18. The medical robot according to claim 16, wherein a 3D mouse is used as the input mechanism, which, in addition to three axis inputs also provides three rotational inputs as input, wherein one axis of the 3D mouse is aligned in a neutral position as a neutral axis coaxially to the end effector axis and/or to a longitudinal axis of the robot arm segment to control the medical end effector and/or the robot arm segment translationally and rotationally in accordance with the input.

19. The medical robot according to claim 16, wherein a force-momentum sensor is used as the input mechanism, the force-momentum sensor providing three axis inputs and three rotation inputs.

20. The medical robot according to claim 16, further comprising a surgical microscope as a visualization device, the surgical microscope having a focal point on a visualization axis.

21. The medical robot according to claim 16, wherein the medical robot has an instrument with an instrument tip on an instrument axis as the medical end effector, and the control unit is adapted to control the robot arm with the instrument in such a way that a position of the instrument tip is maintained during an operating input by the input mechanism.

22. The medical robot according to claim 16, wherein the input mechanism is adapted to select and control an end effector frame and/or a target frame and/or a base frame and/or a world frame and/or a frame defined by a user and/or an image frame as a frame.

23. The medical robot according to claim 16, wherein the input mechanism has six degrees of freedom, wherein translational axes of the input mechanism as axis inputs control translational movements of the robot in a selected frame, and rotational axes of the input mechanism as rotational inputs control rotational movements of the robot in the selected frame.

24. The medical robot according to claim 23, wherein the control unit is adapted to detect exactly three degrees of freedom of the input mechanism as input, wherein either
three translational axes of the input mechanism control three translational movements of the robot in the selected frame, or
three rotational axes of the input mechanism control three rotational movements of the robot in the selected frame.

25. The medical robot according to claim 16, wherein the control unit is adapted to control a single degree of freedom or a combination of degrees of freedom up to five degrees of freedom of the robot based on a number of degrees of freedom between one and five.

26. The medical robot according to claim 16, wherein the medical end effector further comprises a camera with a camera axis, and wherein the camera axis is aligned parallel to the end effector axis.

27. The medical robot according to claim 16, wherein the input mechanism has a puck-shaped or ball-shaped or cube-shaped haptic outer contour as an operating portion.

28. The medical robot according to claim 16, wherein an axis of the input mechanism is arranged parallel to a longitudinal axis of the robot arm segment.

29. The medical robot according to claim 16, wherein an axis of the input mechanism is arranged coaxially to a longitudinal axis of the robot arm segment.

30. The medical robot according to claim 16, wherein the medical end effector is a visualization device having a visualization axis and/or a medical instrument having an instrument axis.

31. A surgical assistance system for use in a surgical intervention on a patient comprising:
the robot according to claim 16; and
a navigation system.

32. A control method for a surgical robot, the control method comprising the steps of:
detecting an input by an input mechanism having an axis that is aligned parallel to an end-effector axis of an end effector and/or to a longitudinal axis of a robot arm segment;
providing the input to a control unit as a control command; and
controlling a position and/or orientation of the end effector and/or of the robot arm segment by the control unit based on the detected input.

33. A computer-readable storage medium comprising instructions which, when executed by a computer, cause the computer to perform the steps of claim 32.
